(19) United States
(12) Patent Application Publication
(10) Pub. No.: US 2021/0121130 A1
(43) Pub. Date: Apr. 29, 2021

(54) ANCHORING SYSTEM FOR A CATHETER DELIVERED DEVICE

(71) Applicant: ENDOTRONIX, INC., Lisle, IL (US)

(72) Inventors: Michael Nagy, Lisle, IL (US); Jerson Martina, Lisle, IL (US); Trace Royer, Lisle, IL (US); Nicholas Chronos, Lisle, IL (US); Thomas Wilschke, Lisle, IL (US); Tyler Panian, Lisle, IL (US); Greg Wolfe, Lisle, IL (US); Omid Forouzan, Lisle, IL (US)

(21) Appl. No.: 17/138,081

(22) Filed: Dec. 30, 2020

Related U.S. Application Data
(63) Continuation-in-part of application No. 15/958,613, filed on Apr. 20, 2018.
(60) Provisional application No. 62/954,791, filed on Dec. 30, 2019.
(60) Provisional application No. 62/487,508, filed on Apr. 20, 2017.
(60) Provisional application No. 62/624,146, filed on Jan. 31, 2018.

(51) Int. Cl. A61B 5/00; A61B 5/06

(52) U.S. Cl. CPC A61B 5/6882; A61B 5/064; A61B 5/0008; A61B 5/6871; A61B 5/6876; A61B 5/6856; A61B 5/6883; A61B 5/6869

(57) ABSTRACT

The present disclosure relates to various anchoring assemblies, systems, and methods for a catheter delivered device or otherwise implantable biomedical sensors. In one instance the anchoring systems of the present disclosure are designed to be used in connection with a biomedical sensor configured to be placed in the various locations within the anatomy of a patient including: a junction of a renal vein and an inferior vena cava, a junction of a jugular vein branch and a subclavian vein branch, a junction of a brachiocephalic vein branch and a superior vena cava, or a junction of an iliac vein branch and an inferior vena cava. In one embodiment, an biomedical sensor and anchoring system can be implanted in an organ of a patient or in an organ to be transplanted within a patient.

### CROSS-REFERENCE TO RELATED APPLICATIONS

This application claims priority to and benefit to U.S. Provisional Patent Application No. 62/954,791 filed Dec. 30, 2019 which claims priority to and benefit of U.S. patent application Ser. No. 15/958,613 entitled “ANCHORING SYSTEM FOR A CATHETER DELIVERED DEVICE,” filed on Apr. 20, 2018, which claims priority to and benefit of U.S. Provisional Application No. 62/487,508 entitled “ANCHORING SYSTEM FOR A CATHETER DELIVERED DEVICE,” filed on Apr. 20, 2017, which also claims priority to and benefit of U.S. Provisional Application No. 62/624,146 entitled “DEVICE AND METHOD FOR DEPLOYING AND SECURING AN IMPLANT TO A VESSEL WALL,” filed on Jan. 31, 2018, which are hereby incorporated by reference in their entirety.

This application is also related to U.S. patent application Ser. No. 14/428,551 entitled “PRESSURE SENSOR, ANCHOR, DELIVERY SYSTEM AND METHOD” filed on Mar. 16, 2015 which claims priority to PCT Patent Application No. PCT/US2013/059769 entitled “PRESSURE SENSOR, ANCHOR, DELIVERY SYSTEM AND METHOD” filed on Sep. 13, 2013 which claims priority to Provisional Patent Application No. 61/701,058 entitled “PRESSURE SENSOR, ANCHOR, DELIVERY SYSTEM AND METHOD,” filed on Sep. 14, 2012, each of which are hereby incorporated by reference in their entirety.

### FIELD OF THE DISCLOSURE

The present disclosure relates to various anchoring systems for a catheter delivered device. In one instance the anchoring systems of the present disclosure are designed to be used in connection with an implant, such as an implant device placed in the vein or artery of a human. In one embodiment, an anchoring system of the present disclosure comprises two anchoring ends, a distal end anchoring structure and a proximal end anchoring structure.

### BACKGROUND

As this use for implantable biosensors has developed and grown, issues regarding intracorporeal fixation of the sensor have come to light. Particularly within blood vessels, the sensor is subjected to a continuous, pulsatile flow. This is a difficult environment in which to secure a sensor or other apparatus reliably without unduly restricting blood flow and/or impairing the vessel wall. Further, some devices require accurate positioning within the body in order to achieve sufficient wireless communication with a device outside the body. One major anatomical area of interest within the human body includes the vessels that circulate blood flow that surround the heart. These vessels may include the pulmonary artery, the superior vena cava, and the inferior vena cava and the related branch vessels. These areas are particularly challenging locations in which to secure an intracorporeal device because, in addition to the above considerations, the vessels can be especially thin-walled, compliant and prone to perforation, have various geometrical configurations, as well as carry blood flow both towards and away from the heart.

Implantable wireless sensors are useful in assisting diagnosis and treatment of many diseases. Some of these sensors may be configured to communicate with wireless sensor readers. Examples of wireless sensor readers are disclosed in U.S. Pat. Nos. 8,154,389, 8,493,187, and 8,570,186 and each are incorporated by reference herein. In particular, there are many applications where measuring pressure from within a blood vessel deep in a patient's body is desired. For example, measuring the pressure in the heart's various arteries and veins may be helpful in optimizing treatment of heart failure and hypertension. In this type of application, an implant may need to be positioned up to 20 cm beneath the surface of the skin. These devices may require a specific implant to provide optimal functionality of the reader/sensor system. An optimal implant for such systems may be configured to transduce pressure into an electrical resonant frequency. Examples of these implants are described in U.S. Pat. No. 9,867,552 entitled “IMPLANTABLE SENSOR ENCLOSURE WITH THIN SIDEWALLS,” and U.S. Utility Ser. No. 14/777,654 entitled “PRESSURE SENSING IMPLANT,” each of which are hereby incorporated by reference herein in their entirety.

Design considerations for an ideal fixation device intended for intravascular fixation are outlined as follows. The fixation device may be passive and maintain a separation distance between the sensor and the vessel wall. Alternatively, the fixation device may be placed against a vessel wall in a particular geometric arrangement for sensing and communication. The implant should have secure attachment against a smooth, slippery surface in the presence of continuous pulsatile flow. The implant should be able to adapt and conform to a compliant surface which may be undergoing radial distention and contraction. The deployed size and outward radial force applied by the device should be sufficient to prevent its migration into vessels or structures (e.g. valves) that would be occluded by the dimensions of the sensor while creating minimal stress concentrations where the fixation device contacts the vessel wall. Alternatively, intracorporeal devices should be designed sufficiently small in size so that when deployed in organs or regions with sufficiently redundant blood flow, the device can endothelialize on its own without harming the organ or the host. Finally, the fixation device should be sufficiently versatile as not to depend, within physiologically relevant ranges, on the size of the vessel in order to maintain its position. The implant should be sufficiently versatile to accommodate a broad range of vessel sizes, curves, random sub-branches, and tortuosity. Otherwise, unintended proximal movement or dislodgement of the fixation device may pose serious health risks that may require surgical intervention.

The device should meet these requirements without damaging or puncturing delicate vessel walls, or without translating, rotating, or becoming dislodged and migrating to a different location in the vessel. Anchors for the device may also be foldable in order to be placed within the vessel with a catheter in a minimally invasive procedure. This is a difficult environment in which to secure an implant or other apparatus reliably without unduly restricting blood flow and/or impairing the vessel wall.

Further, it is a challenge for clinicians to position the implant in a desired location within the vessel of a patient particularly when the location is tied to an allowable vessel size range. Many times it becomes necessary to utilize a still image, recorded video, or live motion video taken via CT scan, fluoroscopy, ultrasound or “quantitative angiography”, etc, to make precise measurements of vessel sizes and configurations with the help of software. This may be particularly true for placement of intracorporeal devices within the superior vena cava or the inferior vena cava as these vessels supply blood flow to the heart. As such, there may be an increased risk of embolus and lower ability to utilize contrast agents for scans of branch vessel anatomy during device placement. These methods require special equipment, added time, and operator skill which may often not be available.

Thus, acute placement and long term stability of an implantable device in a blood vessel or organ is a challenging task. The environment is dynamic and extremely sensitive to disturbances. As such, there are many design considerations associated with fixating the sensor implant within a blood vessel. One consideration is for the sensor and anchoring assembly to be apposed to a specific side of the vessel wall for the safety of the patient and the performance and functionality of the device. An implantable device should be placed where it is intended to land with reduced subsequent rotation or migration. The device should remain stable when exposed to pulsatile blood flow, the changing diameter of a compliant vessel, changing pressures, and several other physiological factors. The device should not exert force that could damage or perforate the vessel wall and it also should not substantially disturb normal blood flow. Finally, the device should remain stable over a diverse range of patient vessel shapes and sizes without clinically traumatizing the vasculature. Any variation of these design factors may degrade electronic communication with the implantable device, cause grave health consequences, or otherwise fail.

Given the above, there is a need in the art for both an improved implant and anchoring system and method of utilizing the same to deliver an implantable device into a blood vessel such as a pulmonary blood vessel or within an organ. The instant disclosure provides an anchor assembly design that is intended to address the above identified problems.

### SUMMARY

The present disclosure relates to various anchoring assemblies and systems for a catheter delivered device or otherwise implantable biomedical sensors. In one embodiment, provided is an anchoring system for a biomedical sensor comprising a biomedical sensor having a housing with a distal end and a proximal end; and an anchoring system comprising a distal anchor and a proximal anchor, where the distal anchor is attached to the distal end of the biomedical sensor and the proximal anchor is attached to the proximal end of the biomedical sensor. At least one of the distal anchor or the proximal anchor is formed with an elongated flexible structure so as to accomplish secure placement of the biomedical sensor upon implantation thereof by a catheter device. At least one distal and proximal anchors is configured to be placed into a retracted position for catheter delivery, and placed in an expanded position for placement within a vessel. At least one anchor is configured to position said housing against a vessel wall, and at least one anchor is configured to adapt to at least one anatomical feature of a vessel to prevent movement of said housing.

The biomedical sensor may be configured to be implanted in a central venous vessel and the biomedical sensor may be designed to be read from the chest of a patient in which the sensor is implanted. The at least one anchor may be a wire wherein said wire may be formed as a generally elongated loop shaped to conform to an inner surface of a vessel. The at least one anatomical feature may be a first vessel segment oriented at an angle with respect to an adjoining second vessel segment and the housing includes a sensor surface configured to be positioned along an axis that is positioned towards a patients chest and configured to communicate with a wireless sensor reader device. The first vessel segment may be a renal vein and said second vessel segment is the inferior vena cava. The housing may be configured to be located in said first vessel segment, and said at least one anchor may be configured to extend into said second vessel segment a distance sufficient to prevent translational or rotational movement of said housing in at least one direction by impeding movement of the housing about said angle formed by said vessel segments.

In one embodiment, a second biomedical sensor may be provided in addition to the initial biomedical sensor, the second biomedical sensor may be configured to be located in a third vessel segment separate from the first and second vessels. The first or initial biomedical sensor and said second biomedical sensor may be configured to communicate wirelessly with each other or with a wireless sensor reader device positioned outside said vessel containing said biomedical sensor and said second biomedical sensor. The anatomical feature may be an intersection of the iliac vessels and the inferior vena cava vessel.

In another embodiment, provided is a method for anchoring an implant inside a blood vessel, comprising the steps of: attaching at least one flexible anchor to a housing, the housing extends along a housing axis; collapsing said anchor to a collapsed configuration and attaching said housing to a catheter; inserting said catheter into a vasculature system and translating said housing to a deployment location; releasing a distal anchor from said catheter and causing said distal anchor to expand; translating the housing to position the distal anchor relative to an anatomical feature within the deployment location; releasing a proximal anchor from said catheter and causing said proximal anchor to expand thereby disconnecting said housing from said catheter, wherein said distal anchor and proximal anchor positions said housing against a wall of said vessel; and removing said catheter. This method may also include the step of referencing an anatomical marker to identify where to position said implant wherein said anatomical marker is a junction of a renal vein and an inferior vena cava, a junction of a jugular vein branch and a subclavian vein branch, a junction of a brachiocephalic vein branch and a superior vena cava, or a junction of an iliac vein branch and an inferior vena cava.

In yet another embodiment, provided is a method for inserting a biomedical sensor and anchoring system for securing same, the method comprising the steps of: attaching a biomedical sensor-anchoring system combination to a catheter where the biomedical sensor-anchoring system combination comprises a biomedical sensor having a housing with a distal end and a proximal end; and an anchoring system comprising a distal anchor and a proximal anchor, where the distal anchor is attached to the distal end of the housing and the proximal anchor is attached to the proximal end of housing. At least one of the distal anchor or the proximal anchor has formed therein an elongated wire structure placed in a retracted position against the catheter so as to accomplish secure placement of the biomedical sensor upon implantation thereof. The method includes the steps of inserting the catheter with the biomedical sensor-anchoring system combination into a desired blood vessel; and implanting the biomedical sensor-anchoring system combination into a desired blood vessel by releasing the biomedical sensor-anchoring system combination from the catheter such that the distal anchor and the proximal anchor are sequentially released from the insertion catheter and expanded to secure placement of the housing in a desired location in the desired blood vessel. The distal anchor may be collapsed over a longitudinal length of the biomedical sensor along the catheter and the proximal anchor is in the retracted position and extended proximally from the proximal end of the biomedical sensor, wherein the step of inserting the catheter may further comprise: inserting a distal end of the catheter into a vessel branch that forms a non-zero angle with an opposing vessel, such that an end portion of at least one of the anchors extend past an apex or intersection formed between said vessel branch and said opposing vessel.

The step of implanting the biomedical sensor may further comprise releasing the distal anchor from the retracted position as the proximal anchor remains in the retracted position wherein the distal anchor extends away from the catheter and abuts against a vessel wall of opposite from said vessel branch in which the catheter is located such that the end portion of the distal anchor is angled towards the opposing vessel along an opposite side of the apex or intersection formed between said vessel branch and said opposing vessel. The step of implanting the biomedical sensor may further comprise translating the catheter proximally to cause the distal anchor to be positioned along the opposing vessel from the proximal anchor and releasing the proximal anchor from the retracted position to abut against a vessel wall along the vessel branch opposite from the distal anchor.

In another embodiment, any of the methods described herein may also include providing a third anchor positioned distally to the distal anchor and the proximal anchor when positioned in the retracted position along the catheter, said third anchor is configured to be released before the distal anchor and the proximal anchor and is configured to extend lengthwise therefrom into an additional vessel segment such as a inferior vena cava vessel. The biomedical sensor-anchoring system combination is configured to prevent migration of the biomedical sensor by inhibiting its ability to migrate within said vessel branch that forms a non-zero angle with the opposing vessel and the inferior vena cava vessel.

In another embodiment, any of the methods described herein may also include inserting a second catheter with a second biomedical sensor-anchoring system into a desired blood vessel wherein the biomedical sensor and second biomedical sensor may be configured to wirelessly communicate with a device outside a chest of a patient.

In one embodiment, provided is an anchoring system for a biomedical sensor comprising: a biomedical sensor; and an anchoring system comprising an elongated hollow tube having a distal end and a proximal end, where the biomedical sensor is configured to be attached within the elongated hollow tube. The biomedical sensor may be positioned adjacent to at least one of the distal end or the proximal end of the elongated hollow tube and the elongated hollow tube is configured to be inserted into an organ or anatomy of a patient to provide a stent or shunt to said organ. The proximal end of the elongated hollow tube may be configured to be in communication with a fluid inlet of an organ and the distal end of the elongated hollow tube is configured to be in communication with a fluid outlet of said organ wherein the elongated hollow tube allows for fluid communication between the inlet and the outlet to allow for fluid bypass of the organ. The organ may be a liver and the elongated hollow tube may be used as a shunt between a portal vein and a hepatic vein. The biomedical sensor may be configured to wirelessly communicate at least one of the following data points: pressure, temperature, GPS location, time, elevation, acidity, salinity, chemical composition, flow rate, and signal strength.

In another embodiment, provided is a method for implanting a biomedical sensor into an organ to be transplanted into a patient, the method comprising the steps of (i) explanting the organ from a donor; (ii) implanting the organ with a biomedical sensor, wherein said biomedical sensor is a wireless sensor configured to communicate wirelessly with a wireless reader device; and (iii) surgically implanting the organ with the biomedical sensor into a receiving patient. The biomedical sensor may include proximal and distal anchors and may be implanted into a vasculature system of the organ using a catheter. The biomedical sensor may be provided with at least two anchors configured to extend into a vessel branch of said vasculature system wherein said vessel branch that forms a non-zero angle with an opposing vessel, such that an end portion of at least one of the anchors extend past an apex or intersection formed between said vessel branch and said opposing vessel, said anchors being collapsible when tied to said catheter and releasable from said catheter. Additionally, said biomedical sensor may be implanted into said organ by direct suture or staple. Optionally, the biomedical sensor may be implanted into a vestigial vasculature of a receiving patient near the point where it will connect to said transplanted organ, prior to implantation of said transplanted organ. The organ may be selected from the following: heart, lung, kidney, spleen, stomach, pancreas, heart, skeletal joints, and liver.

### BRIEF DESCRIPTION OF THE DRAWINGS

FIG. 1 is a perspective view of a known implant;

FIG. 2 is a photographic illustration of a biomedical sensor or implant and an anchoring structure, attached to the distal end of a delivery catheter according to an embodiment of the present disclosure in a state ready for insertion into a patient and/or individual;

FIG. 3A is a photographic illustration of an embodiment of a biomedical sensor or implant and an anchoring structure attached thereto in a state where the anchoring structure is in its expanded state as would be the case once placement occurs in a desired blood vessel;

FIG. 3B is a schematic illustration of an embodiment of a biomedical sensor or implant and an anchoring structure attached thereto in a state where the anchoring structure is in an expanded state as would be the case once placement occurs in a desired blood vessel;

FIG. 4 is a photographic illustration of a biomedical sensor or implant and an anchoring structure attached thereto in a state where the anchoring structure is in an expanded state in a 14 mm blood vessel;

FIG. 5A is an end view of an embodiment of a biomedical sensor or implant with an anchor assembly including a distal anchor having an elongated and angled orientation and a proximal anchor having three lobes in accordance with the present disclosure;

FIG. 5B is a perspective view of the embodiment of the biomedical sensor or implant and anchor assembly of FIG. 5A;

FIG. 6A is a photographic illustration of a biomedical sensor or implant of FIGS. 5A and 5B positioned within a model of a pulmonary artery;

FIG. 6B is a photographic illustration of the a biomedical sensor or implant of FIGS. 5A and 5B positioned within a model of a pulmonary artery;

FIG. 7A is a photographic illustration of the a biomedical sensor or implant of FIGS. 5A and 5B positioned within a model of a pulmonary artery;

FIG. 7B is a photographic illustration of the a biomedical sensor or implant of FIGS. 5A and 5B positioned within a model of a pulmonary artery;

FIG. 8 is a schematic illustration of an embodiment of the present disclosure where a biomedical sensor or implant and an anchoring structure attached thereto are in a state where the anchoring structure is in an expanded state.

FIG. 9 is a schematic illustration of several branches of the pulmonary artery;

FIG. 10 is a schematic cross-sectional view of a biomedical sensor or implant positioned within the body of a patient and in communication with a wireless sensor reader or reading device;

FIG. 11A is a schematic illustration of various vessels of the human anatomy and an embodiment of a biomedical sensor or implant with an anchor assembly positioned within the inferior vena cava;

FIG. 11B is a schematic illustration of various vessels of the human anatomy and an embodiment of a biomedical sensor or implant with an anchor assembly positioned within the vena cava;

FIG. 12A is a schematic illustration of an embodiment of a biomedical sensor or implant being positioned at the junction of the common iliac veins of a patient;

FIG. 12B is a schematic illustration of an embodiment of a biomedical sensor or implant being positioned at the junction of the common iliac veins of a patient;

FIG. 12C is a schematic illustration of an embodiment of a biomedical sensor or implant being positioned at the junction of the common iliac veins of a patient;

FIG. 12D is a schematic illustration of an embodiment of a biomedical sensor or implant being positioned at the junction of the common iliac veins of a patient;

FIG. 13 is a schematic illustration of an embodiment of a biomedical sensor or implant positioned within the superior central veins of a patient;

FIG. 14 is a schematic illustration of an embodiment of a guidewire used to place a biomedical sensor or implant positioned within an anatomy of a patient such as the liver;

FIG. 15 is a schematic illustration of an embodiment of biomedical sensor or implant positioned within a shunt within an anatomy of a patient;

FIG. 16 is a schematic illustration of an embodiment of a sensor device or implant positioned within an organ prior to being transplanted within an anatomy of a patient;

FIG. 17 is a flow chart of an embodiment of a method of implanting a biomedical sensor into a patient; and

FIG. 18 is a flow chart of another embodiment of a method for implanting a biomedical sensor into a patient.

### DETAILED DESCRIPTION

The present disclosure relates to various anchoring systems for a catheter delivered device. In one instance the anchoring systems of the present disclosure are designed to be used in connection with a biomedical sensor or implant to be placed within the cardiovascular system. The terms biomedical sensor and implant are used interchangeably herein for convenience, however, it is understood that a biomedical sensor includes a sensor and an implant may optionally also include a sensor such as the type of sensors as described herein. In one embodiment, an anchoring system of the present disclosure comprises two anchoring ends, a distal end anchoring structure and a proximal end anchoring structure, where at least one of the distal or proximal anchoring structures has a clover-shaped structure formed by at least three lobes. In another embodiment, the distal end anchoring structure has an elongated and angled orientation relative the implant body. In another embodiment, both the distal and proximal anchoring structures have a clover-shaped structure formed by at least three lobes.

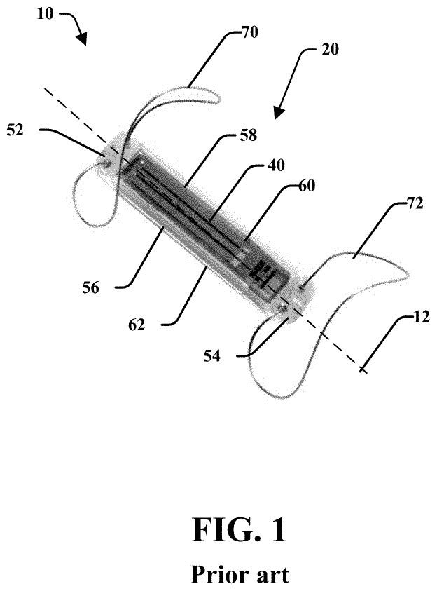

FIG. 1 illustrates a prior art implant 10 that includes a housing 20 that includes an oblong, narrow, rectangular shape that extends along a housing axis 12, although the housing may have various shapes and geometry. The dimension of the housing 20 may be generally cuboid and may define a cavity therein. The housing side walls may be of specific dimensions and proportions to each other. For example, the housing may have four side walls 52, 54, 56, and 58, a top wall 60 and a bottom wall 62. The housing 20 may be made of a hermetic, strong, and biocompatible material, such as ceramic. The examples illustrate a cuboid housing, but other shapes and configurations may be used, such as cylindrical housings, prism-shaped housings, octagonal or hexagonal cross-sectioned housings, or the like. A sensor 40 is positioned along the top wall 60 and is attached to an antenna coil as well as other electronic components that may be positioned within the housing of the implant. The sensor 40 as well as the antenna coil and internal electronics may be positioned along a sensor axis 42 that extends generally normal relative to the implant 10 wherein the sensor 40 along the top surface 60 may be exposed to blood flow and pressure within the vessel once positioned within a patient. A distal anchor 70 and a proximal anchor 72 opposite the distal anchor may extend from the top surface of the implant 10. The anchors may fixate the implant 10 in a desired position in the body of the patient.

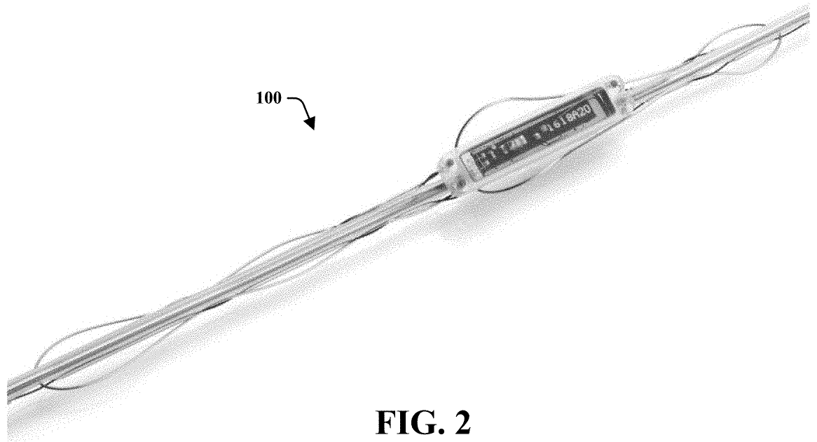

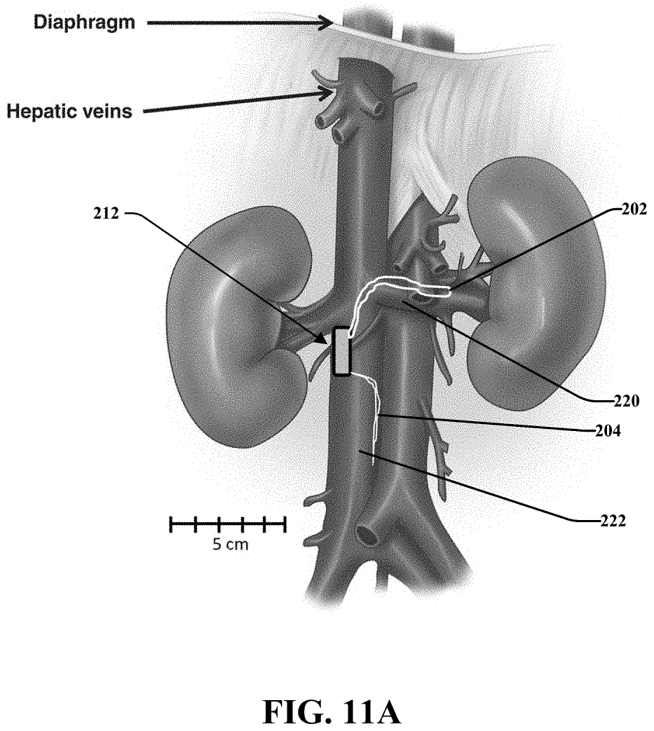

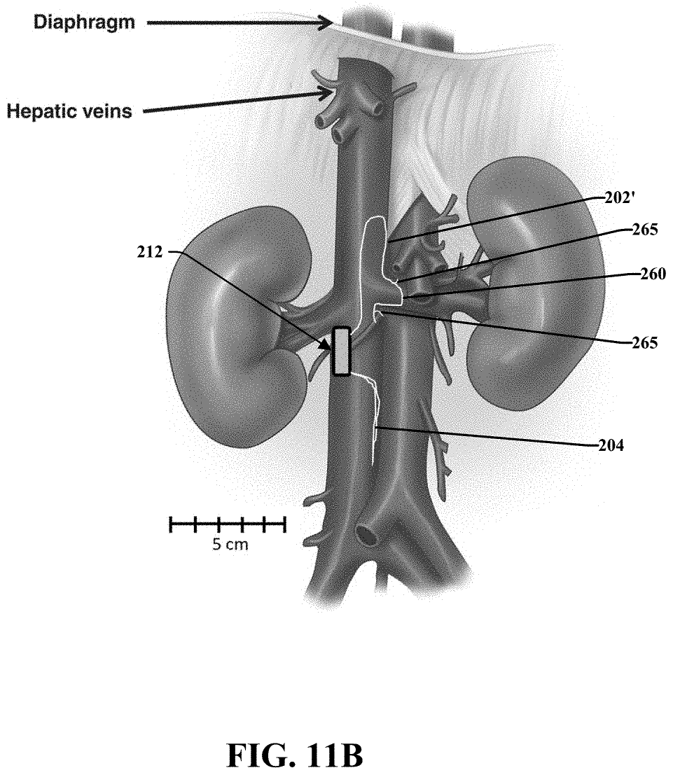

FIGS. 2-10 disclose various embodiments of an anchoring system while FIGS. 11-16 disclose various embodiments of implanting systems and methods according to the present disclosure. FIG. 2 depicts an embodiment of an anchoring assembly 100 with its anchors attached to the catheter in a retracted state and the housing positioned thereon ready for insertion into a patient and/or individual by, for example, the catheter in a minimally invasive procedure.

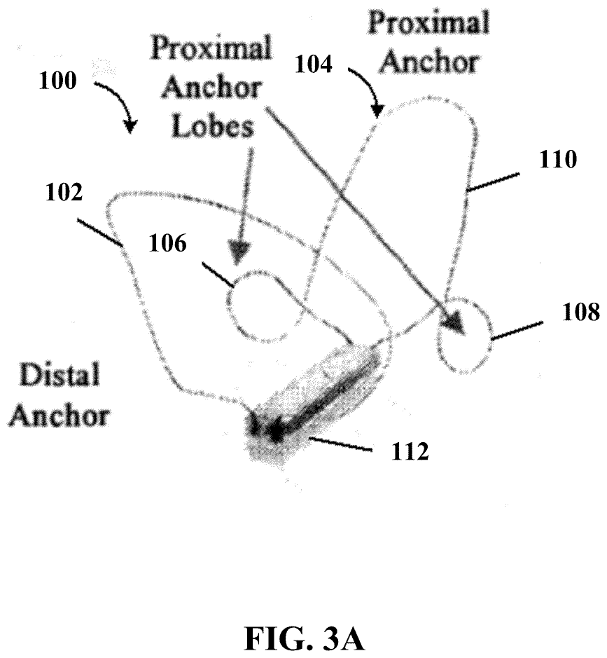

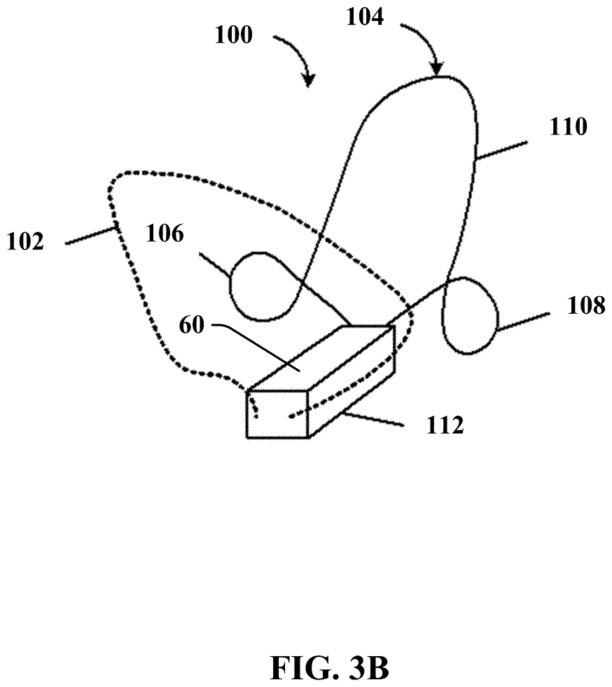

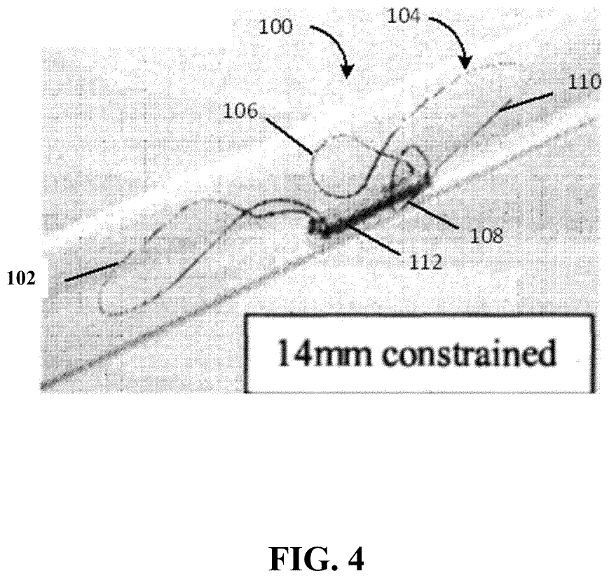

As illustrated by the embodiments in FIGS. 3A-3B, the anchoring assembly 100 comprises two anchoring ends, a distal anchoring structure 102 and a proximal anchoring structure 104, where at least one of the distal or proximal anchoring structures 102/104 has a clover-shaped structure formed by at least smaller two lobes 106 and 108 located on either side of a larger lobe 110. Located in between the distal anchoring structure 102 and the proximal anchoring structure 104 is a suitable implant sensor 112, such as the implant 10 illustrated by FIG. 1. FIG. 4 depicts the present invention in a state where the anchoring structure is in an expanded state in a 14 mm blood vessel, e.g., a pulmonary blood vessel. The term “distal” herein will be referred to as meaning away from a catheter handle while the term “proximal” herein will be referred to as meaning towards a catheter handle.

The distal anchoring structure 102 and the proximal anchoring structure 104 may extend from a top surface 60 of the implant 10. Notably, the top surface 60 may include a sensor 40 as illustrated by FIG. 1. Alternatively, the implant 10 may include an actuator such as one that may be selected from among the following: neurostimulation, cardiac pacing, electrical stimulation, drug elution and the embodiments of the implant and anchoring system is not limited as to the type of sensor that may be utilized. Furthermore, the anchoring structures may comprise two individual shape set nitinol wires. As illustrated by FIGS. 3A-3B, the two wires comprise a distal wire and a proximal wire, where one anchor wire 102 is attached to the distal portion of a top surface 60 (spade shape, see FIGS. 3A-3B) of the implant 112 and the other anchor wire 104 is attached to the proximal portion of the top surface 60 (club shape, see FIGS. 3A-3B). Both anchors 102/104 can be collapsed down and attached to a delivery catheter via “release wires” or other mechanism like a shroud. The implant 112 and anchors 102/104 can be introduced into the human vasculature in the collapsed position and expanded to place the implant within a desired location of a vessel.

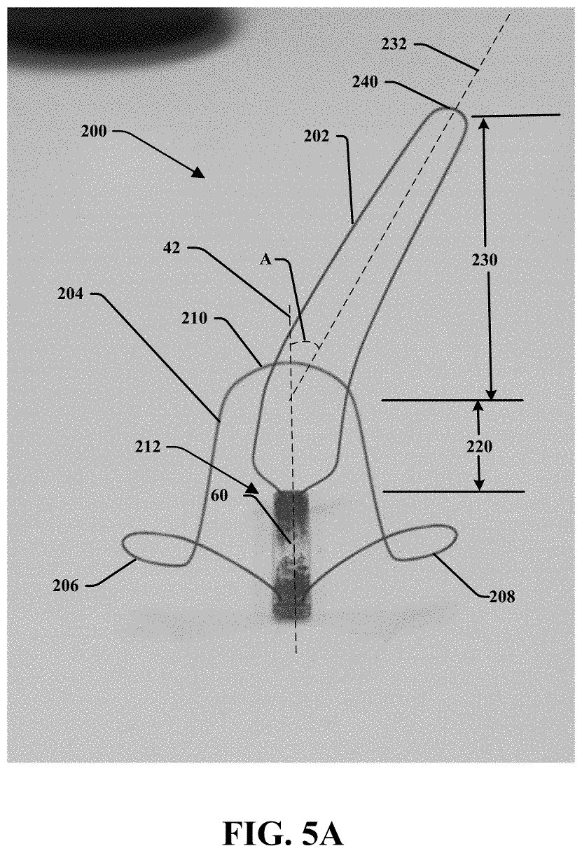

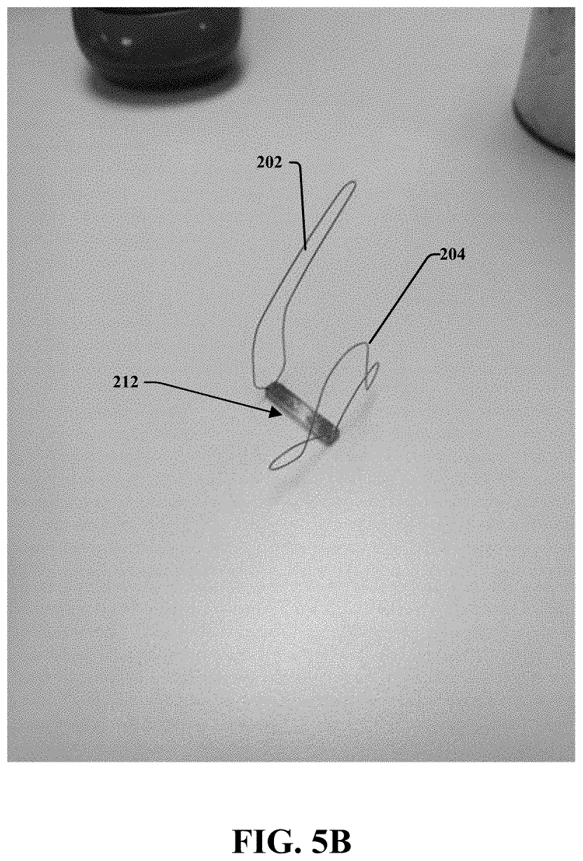

FIGS. 5A-5B illustrate an embodiment of an anchor assembly 200 with a distal anchoring structure 202 and a proximal anchoring structure 204. The distal anchoring structure has a wire shape with an elongated and angled orientation relative to the implant 212. The proximal anchoring structure 204 includes a wire that is shaped as a clover-shaped structure formed by at least smaller two lobes 206 and 208 located on either side of a larger lobe 210. Located in between the distal anchoring structure 202 and the proximal anchoring structure 204 is a suitable implant 212 such as one illustrated by FIG. 1.

The distal anchoring structure 202 and the proximal anchoring structure 204 may extend from a top surface 60 of the implant 212. Notably, the top surface 60 may include a sensor 40 that is attached to an antenna coil within the cavity of the implant housing as illustrated by FIG. 1. Furthermore, the anchoring systems of the present invention may comprise two individual shape set nitinol wires. As illustrated by FIGS. 5A-5B, the two wires comprise a distal wire and a proximal wire, where one anchor wire 202 is attached to the distal portion of a top surface 60 (elongated and angled, see FIGS. 5A-5B) of the implant 212 and the other anchor wire 204 is attached to the proximal portion of the top surface 60 (club shape, see FIGS. 5A-5B). Both anchors 202/204 can be collapsed down and attached to a delivery catheter via “release wires” or other mechanism. The implant 212 and anchors 202/204 can be introduced into the human vasculature in the collapsed position.

In the expanded position, the three-lobed proximal anchor 204 may radially expand to abut the inner wall of the vessel. Lobes 206 and 208 may expand outwardly from the implant 212 while lobe 210 may extend upwardly from the implant 212. These three lobes may radially abut against the inner wall of the vessel and may be arranged to abut against vessels of various sizes. The elongated and angled distal anchor 202 may include a slender configuration that may include a base portion 220 that may extend upwardly and slightly outwardly from the width of the implant 212 and an elongated portion 230 that may extend from the base portion 220 at an angle that includes a gradual taper until it ends at end portion 240. The elongated portion 230 may extend along elongated axis 232 wherein the elongated axis 232 may be positioned angularly relative to the sensor axis 42 as identified in FIG. 5A. The elongated axis 232 may intersect the sensor axis 42 at angle A wherein angle A may be about 20 degrees to about 40 degrees, or more particularly may be about 30 degrees. The elongated portion 230 may be over twice the length of the base portion 220. The slender elongated angle configuration may allow the distal anchor 202 to extend within a branch vessel of the pulmonary artery (“PA”) and may correctly position the implant 212 to allow the sensor axis 42 to extend towards the chest of a patient. Further, the configuration of the anchors 202, 204 may be arranged to allow the catheter or other delivery device to deploy the implant 212 and the anchor assembly 200 with enough room to allow the catheter to be removed without bumping or rubbing against the implant 212 or the anchors 200 in which it may otherwise move or rotate the implant from its desired position.

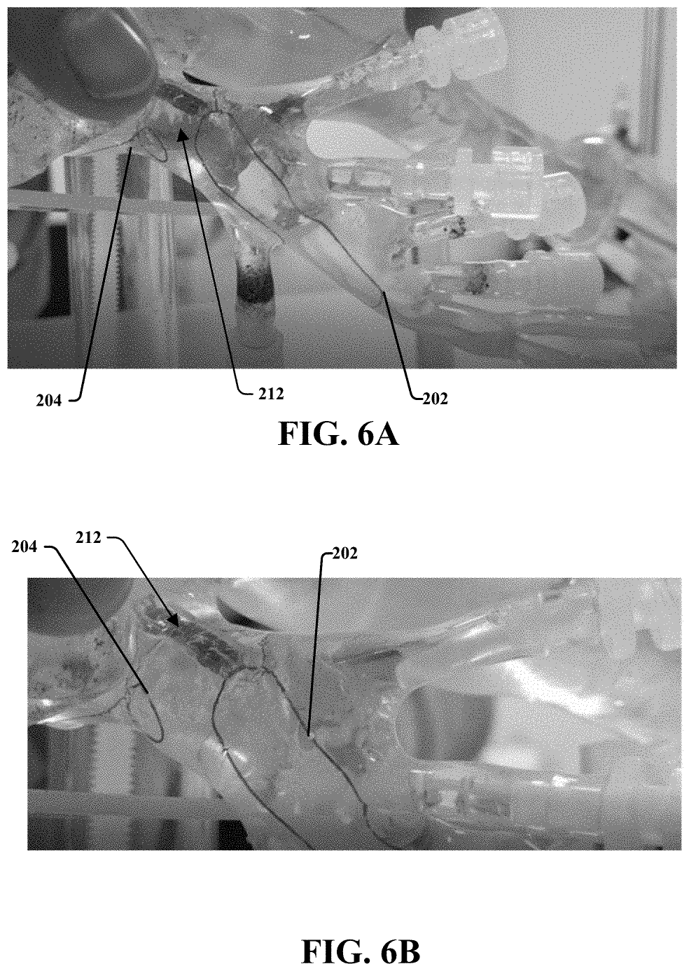

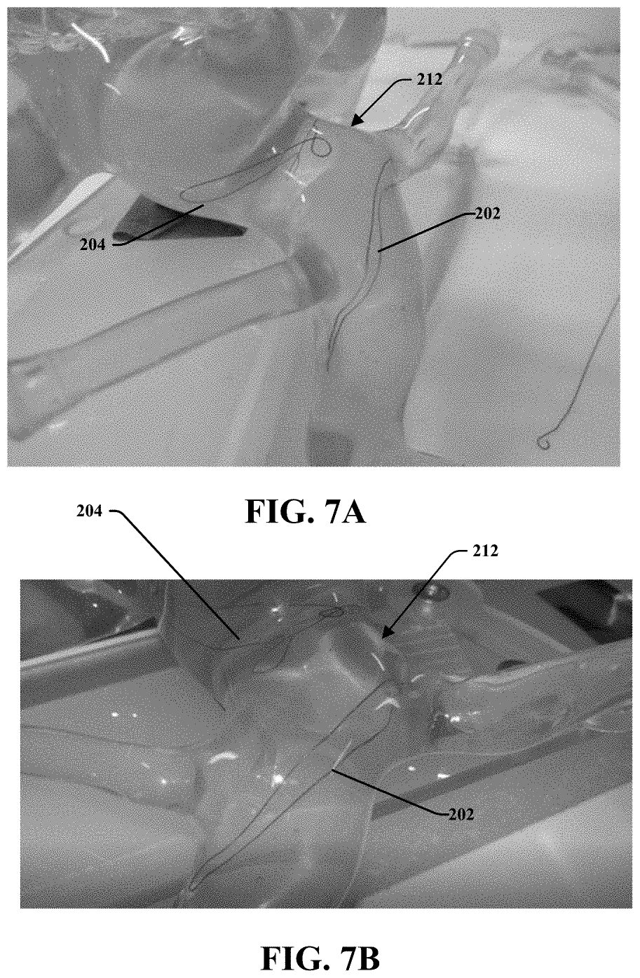

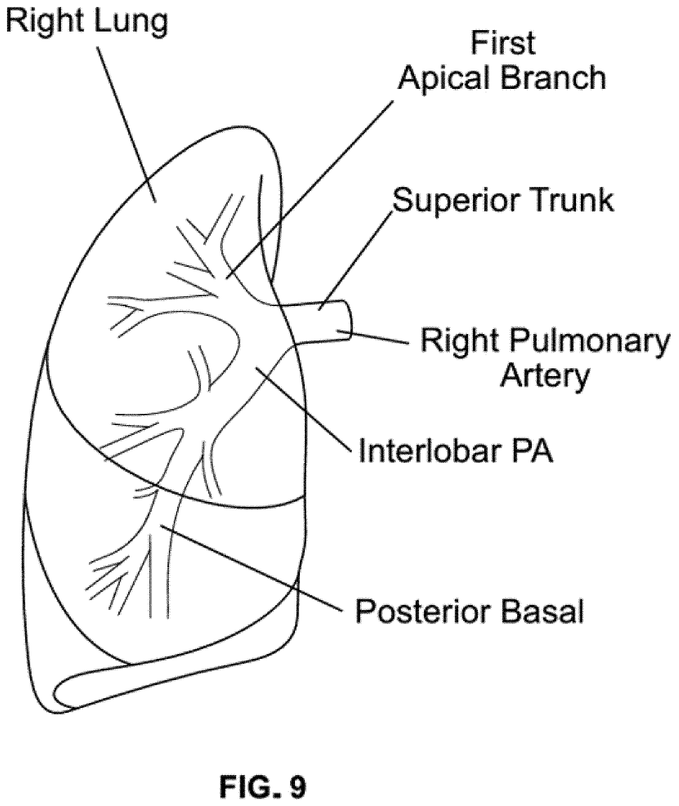

It has been found that the elongated and angled configuration of the distal anchoring structure 202 may provide various benefits which may allow clinicians to deploy the implant at an exact location and orientation with a reduced risk of translation or rotation once deployed. In one embodiment, the implant 212 with the distal anchor 202 may be placed in the right main trunk of the PA. As such, clinicians may be able to position the implant within the PA without having to rely on CT scans or quantitative angiography. Instead, in an embodiment, the clinician may reference the first apical branch of the right main trunk of the PA as an anatomical marker to identify where to position the implant 212 in which the elongated distal anchor 202 may be positioned. FIG. 9 is a labeled sketch of the right pulmonary artery wherein the first apical branch is positioned adjacent the superior trunk of the right PA. It should be noted that a wide variety of vessel anatomy exists between patients. This includes differences in size and number of branches. Despite all this variation, the right PA main trunk has an anatomical feature that is present in nearly all patients which the clinician may reference for implant placement: a sharp downturn from the right interlobar segment into the right posterior basal segment, see FIG. 9. The elongated and angled distal anchoring structure 202 may allow the implant 212 to self-correct its position within the vessel. As illustrated by FIGS. 6A-6B and 7A-7B, the distal anchor 202 may be positioned to extend deep into the right posterior basal segment branch of the PA while the proximal anchor 204 may be positioned upstream in the interlobar PA segment. The distal anchor 202 may contact the vessel wall at or near the end portion 240 but it does not need to. Further, it does not need to contact along other radial positions along the base portion 220 or near the base portion 220 along the elongated portion 230, although in some patients it may do so. This may help prevent the implant from translating and rotating and allow the implant to be permanently located therein.

These anchors may allow for ease of implant placement as the distal anchor may be long enough so that when the catheter is removed, there is very little chance of migration into a side branch of the PA. The embodiment may also provide an anatomical landmark facilitating location of the target implant site, that may be easily identified by basic angiography and may allow a clinician to align the implant such that is just distal from the superior trunk takeoff and proximal to the downturn of the PA. The disclosure may further prevent unwanted rotation due to: the spring force nature of the anchor, delivery system rubbing against the implant during removal, and patient coughing or other patient movement. The angle that the posterior basal makes with respect to the chest skin surface may ensure that the implant itself or more particular the housing of the implant with the sensor thereon assumes an angle towards the chest of a patient that is optimal for wireless or RF communication. The angle may ensure that the implant (along with the sensor and its top surface 40) faces the chest surface when the distal anchor 202 is placed into the posterior basal segment of the PA.

Further, if there is an unintentional deployment that is too distally positioned in the PA, the distal anchor 202 may still fit within the right posterior basal segment. If there is an unintentional deployment too proximally positioned, the distal anchor 202 may act to “pull” the implant 212 in the distal direction. In the event that the lobes of the proximal anchor 204 may migrate due to the spring force action, the downturn of the distal anchor 202 in the posterior basal segment may prevent it from translating as the elongated distal anchor will be generally prevented from “turning the corner” as the device moves proximally. Further, if there is migration of the implant 212 distally, the housing and distal anchor 202 may form an angle that prohibits them from making the turn. As such, the implant 212 includes self-adjusting properties in this anatomical location within the pulmonary artery.

The two anchors may act to hold the bottom surface 62 of the implant 212 against the vessel wall with the sensor 40 and top surface 60 away from the vessel wall. Because the posterior basal segment is relatively thin, the implant may not sit any other way. The proximal anchor may hold the implant body against the vessel wall by itself without help from the distal anchor 204. The distal anchor may utilize its length relative to the implant to prevent rotation, by staying in the downturn. Additionally, it may prevent unintended interactions with other branches of the PA. The distal anchor 202 may not include loops and may be too straight and long to migrate into side branches easily.

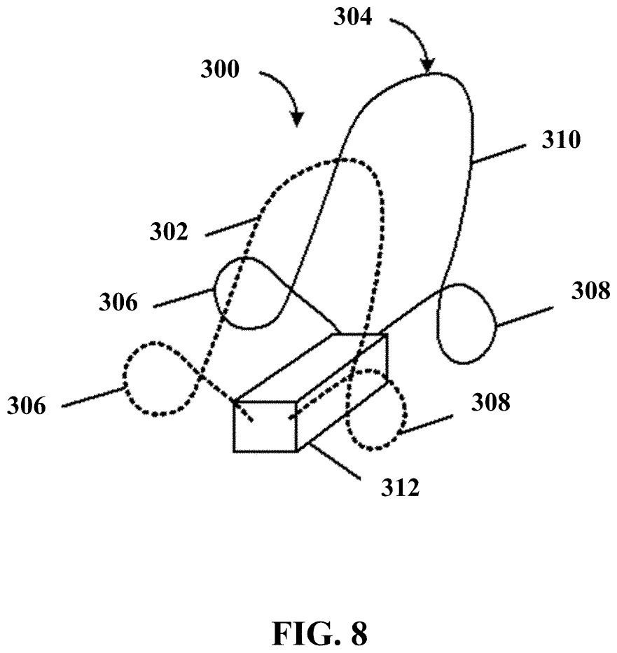

In another embodiment as is illustrated in FIG. 8, an anchoring system 300 comprises two anchoring ends, a distal end anchoring structure 302 and a proximal end anchoring structure 304, where both the distal or proximal anchoring structures 302/304 have clover-shaped structures formed by at least two sets of smaller lobes 306 and 308 located on either side of a larger lobe 310. Located in between the distal end anchoring structure 302 and the proximal end anchoring structure 304 is a suitable implant 312, which may contain a sensor.

The anchoring structures of FIGS. 3A-3B, 5A-5B, and 8 are illustrated in the expanded position and it is understood that the anchors may be positioned in a collapsed or retracted position when attached to a catheter or other type of delivery device, such as that shown in FIG. 2. Notably, other anchor configurations and shapes may be implemented, including a different number of anchors (other than two); different locations of anchor attachment to the housing; anchors which attach to the housing at one point, or more than two points; anchors that extend under the housing, around it, or laterally to the sides. The anchors may be formed as loops which anchor the implant to body structures or within a vessel using spring force. The anchors may be made of nitinol, stainless steel, polymer, or any material which is biocompatible and extrudable. The anchors may be made of a combination of materials, such as nitinol with a platinum core. The anchors may be configured to fold down during the implantation procedure to allow easy ingress to the deployment location. The anchors may be configured to be tied down to a delivery system, such as a catheter, for minimally invasive ingress to the implant deployment site. The anchors may be designed to deploy from their tied-down configuration to their open configuration when an operator actuates a control on the proximal end of the delivery system. The control may include release wires that are pulled from the proximal end either directly or with help from a mechanical handle. The anchors may be coated with a material to increase lubricity.

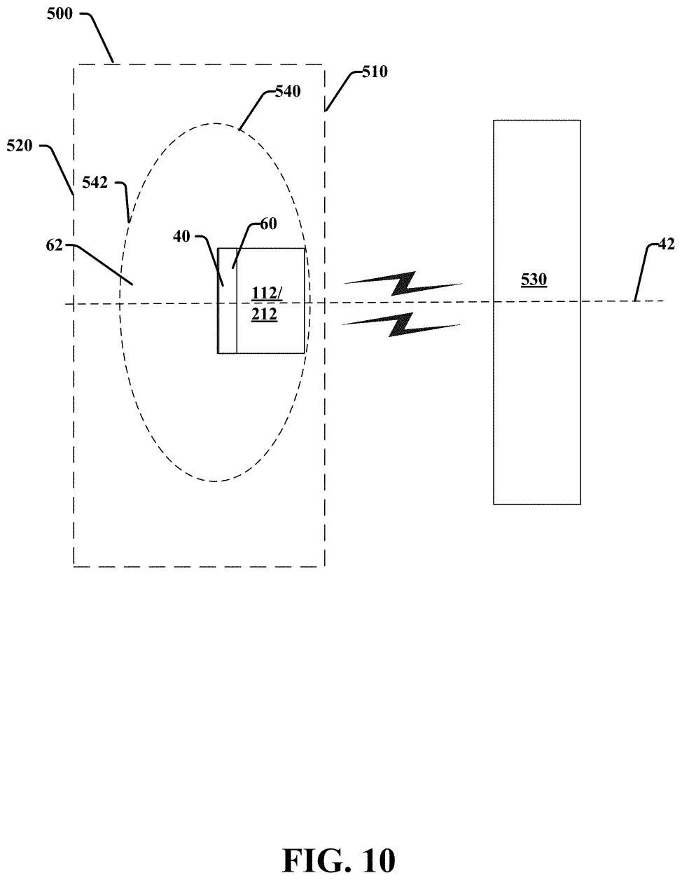

The anchors may be positioned within the vessel at a desired location and caused to expand in the illustrated expanded positions as illustrated in FIGS. 4, 6A-B, and 7A-B. In these embodiments, as illustrated by FIG. 10, it may be desirable to position the implant 112, 212 within a vessel 540 with the top surface 60 and the sensor 40 aligned along the sensor axis 42 directed through the chest 510 of the patient and wherein the top surface 60 may be spaced from an inner wall 542 of the vessel 540. The configuration of the disclosed anchors may make this position possible as the aligned direction would allow the patient to utilize a reader device 530 to be positioned on or near the chest in proximity to the implant while also being directionally aligned with the top surface 60 and sensor 40. FIG. 10 schematically illustrates a cross sectional view of an implant 112, 212 positioned within a body 500 of a patient wherein the top surface 60 and sensor 40 thereon may be directed towards the center of a blood vessel 540. The implant 112, 212 may be located on the side of vessel 540 such that its distance from the wall of the chest 510, and hence from external reader 530, is minimized. The sensor 40 on the top surface 60 may be aligned along the sensor axis 42 that extends through the chest 510 and a user may allow the reader device 530 to be placed in alignment with the sensor axis 42 to wirelessly communicate with the implant through the chest 510 of the patient. The configuration of the anchor assemblies may allow for the implant to be placed in the desired location, so that a patient having the implant may be able to hold reader device 530 and take his own readings from the implant without the assistance of others. The above embodiment may also be applied to the implant of FIG. 8.

During the deployment of the implant 112/212/312 with its and anchoring assemblies 100/200/300, the anchors 202, 204 may be deployed sequentially when the release wires are retracted. Once an anchor is free and/or fully released, the anchor may utilize nitinol's shape memory or superelastic property and instantly attempt to return to its initial shape set shape within the vessel. The distal anchor 202 may deploy first, pushing the distal end of the implant off the delivery catheter and onto the target position along the vessel wall. Next the proximal anchor 204 may deploy, pushing down the proximal end of the sensor body or housing (the ‘implant’) 212 along the vessel wall target and engaging the two side lobes. Although stated in terms of implant or biomedical sensor, the above may be applied to any of the embodiments described herein.

Furthermore, the anchoring systems of the present invention comprise two individual shape set nitinol wires. As discussed above, the two wires comprise a distal wire and a proximal wire, where one anchor wire 102 is attached to the distal end of the implant 112 and the other anchor wire 104 is attached to the proximal end. Both anchors 102/104 can be collapsed down and attached to a delivery catheter via “release wires.” The implant 112 and anchors 102/104 may be introduced into the human vasculature through a 14 Fr introducer. The anchors 102/104 may be deployed sequentially when the release wires are retracted. Once an anchor is free and/or fully released, the anchor utilizes nitinol's shape memory or super elastic property and instantly attempts to return to its set shape within the vessel. The distal anchor deploys first, pushing the distal end of the implant off the delivery catheter and onto the target position along the vessel wall. Next the proximal anchor deploys pushing down the proximal end of the sensor body along the vessel wall target and engaging the two side lobes which provide the most radial force and the largest deterrent to proximal migration and rotation. Although stated in terms of implant 100, the above may be applied to any of the embodiments described herein, including implants 212 and 312.

In one embodiment, the overall implant and anchoring structures are sized such that the anchoring system allows the implant to be placed in a proximal segment of the pulmonary artery. The proximal placement optimizes communication in one direction (dorsal or ventral), and may allow communication with the device to occur from the chest instead of the back. The anchoring system of the present invention is designed to keep maximum vessel contact and remain stable over a large range of vessel sizes as compared to other devices known to those of skill in the art. The anchoring system of the present disclosure is designed to withstand any forces imposed by contact with the delivery catheter during catheter retraction, which is a well-documented procedure risk for devices designed with anchoring system failing to possess the various physical structures of the present disclosure. For example, if the insertion catheter snags the tip of the proximal anchor, the force provided by the proximal anchor lobes increases to mitigate proximal movement.

As would be apparent to those of skill in the art, the use of the labels proximal and distal are for convenience sake and could be interchanged such that in the embodiment of FIGS. 3A-B, the distal end of the anchoring system 100 would have the clover-shaped structure formed by at least three lobes 106, 108 and 110. Such a change in orientation could be dictated by the environment and/or blood vessel in which the anchoring system and sensor device of the present disclosure are to be implanted in. The same may be applied to any of the embodiments described herein, including implants in FIGS. 5A-B and FIG. 8.

Regarding the nitinol wires utilized in the embodiments of the present disclosure, such wires are well known in the art and as such a detailed discussion herein is omitted for the sake of brevity. However, as is known to those of skill in the art, nitinol is formed from at least one nitinol alloy, where such alloys exhibit two closely related and unique properties: shape memory effect (SME) and superelasticity (SE; also called pseudoelasticity, PE). Shape memory is the ability of nitinol to undergo deformation at one temperature and then recover its original, un-deformed shape upon heating above its “transformation temperature”. Superelasticity occurs at a narrow temperature range just above its transformation temperature; in this case, no heating is necessary to cause the un-deformed shape to recover, and the material exhibits enormous elasticity, some 10 to 30 times that of ordinary metal. Given nitinol's biocompatibility it is well suited for use in biomedical devices and/or implants. Regarding the relationship between smaller lobes 106/108 and 206/208 and larger lobe 110 and 210 of the multi-lobed anchoring structures of the present disclosure, it should be noted that the larger lobe should have an overall length of at least 200 percent the length of the smaller lobes.

Additionally, it has been found that various configurations of the anchoring structure and delivery catheter may assist with positioning a biomedical sensor or implant 112/212/312 in various different anatomical positions within a patient or within a transplantable organ as will be discussed more fully with respect to FIGS. 11A-16. In particular, it is contemplated that an implant 212 may be positioned within the vena cava region of the anatomy wherein the vena cava region (“VC”) includes the superior vena cava (“SVC”), the inferior vena cava (“IVC”) and associated branch vessels, collectively the Central Venous System (CVS).

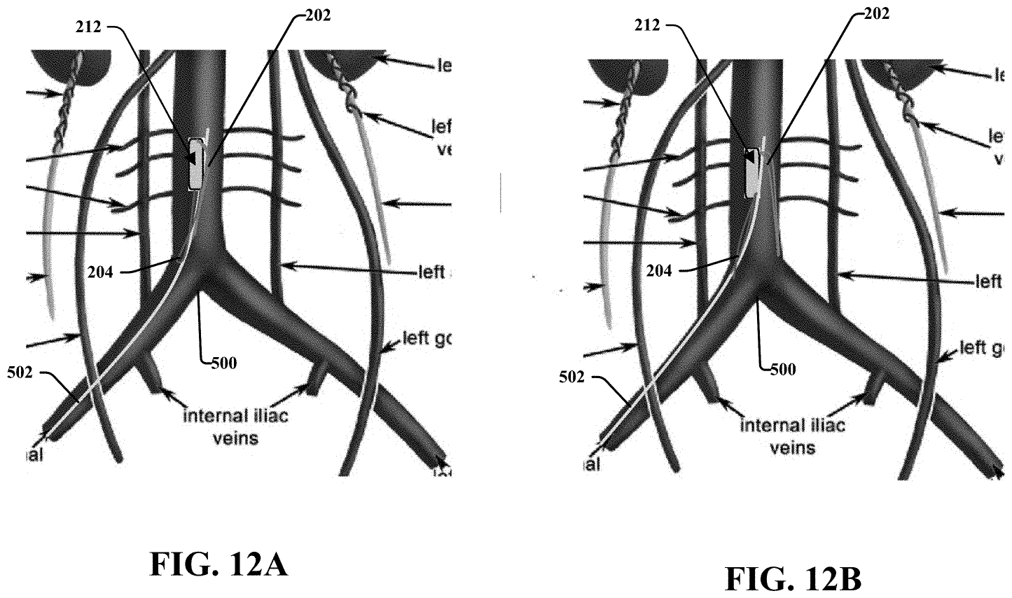

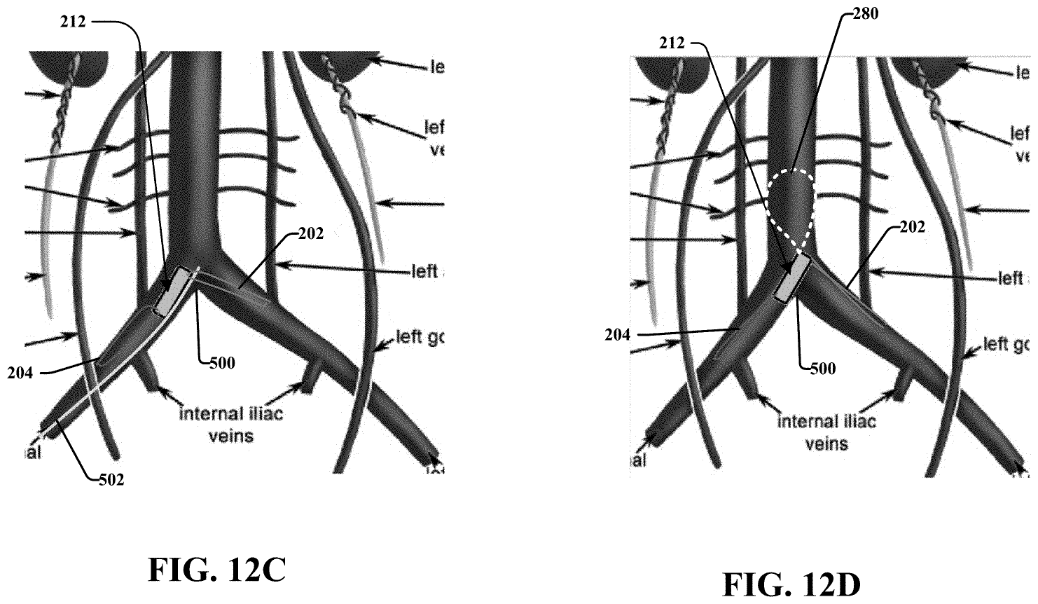

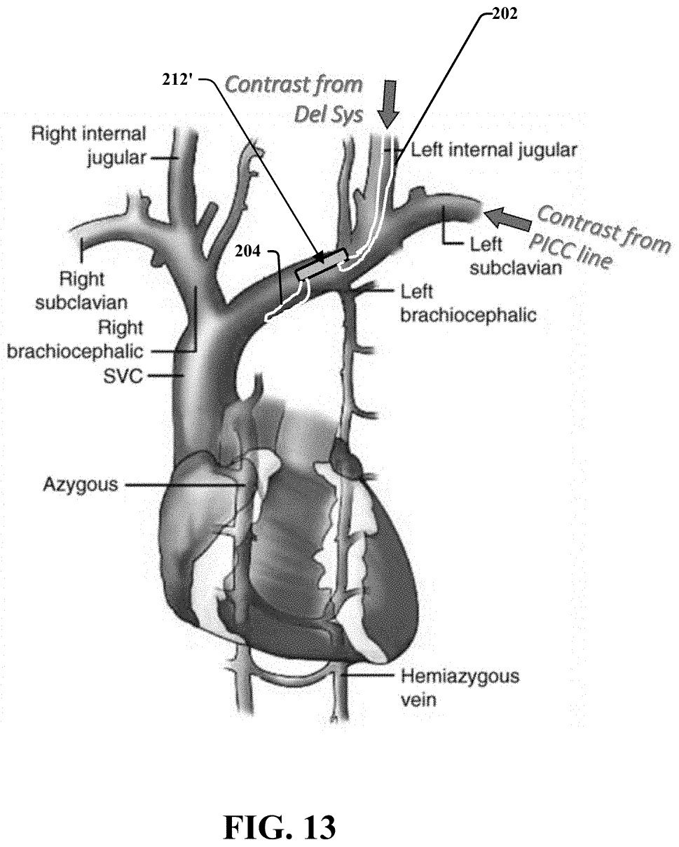

Notably, as the vena cava vessels supply blood to the heart and the directional flow of blood prompts certain considerations for designing an implant, anchoring structure, method and system for delivering and placing such an implant within the CVS. The embolus risk severity is higher in the CVS because flow goes towards the heart and may have a slower flow rate then flow in vessels exiting the heart. As discussed above, various different configurations of the anchoring assembly are contemplated and may assist to place the implant within a desired anatomical feature that exists within the inferior CVS such as the renal vessels (FIGS. 11A and 11B) or the iliac vessels (FIGS. 12A-12D). Other embodiments contemplate that the anatomical feature may also be within the superior CVS such as the brachiocephalic vessels, the subclavian vessels or even within the jugular vessels (FIG. 13). However, it is also contemplated that the various features of the embodiments contemplated herein may be combined or used in various combinations and this disclosure is not limiting. Notably, this disclosure also contemplates an anchoring system for an implant or biomedical sensor and method for use with various organs wherein the anchoring system may allow for a biomedical sensor or implant to be placed into a stent, shunt, or other device prior to surgical insertion of that device into an organ, or may also allow for a biomedical sensor or implant to be placed into the vasculature of an organ to be surgically transplanted into a patient. Here, the biomedical sensor or implant is transplanted into the organ prior to implantation of the transplanted organ into the host patient.

It should be noted that a wide variety of vessel anatomy exists between patients. This includes differences in size and number of vessel branches. Despite all this variation, the IVC has an anatomical feature that is present in nearly all patients in which the clinician may reference for implant placement: a sharp branch traverse to the right and left of the IVC into the right and left kidneys, see FIGS. 11A and 11B. It has been found that the elongated and angled configuration of the distal anchoring structure 202 may provide various benefits which may allow clinicians to deploy the implant at an exact location and orientation with a reduced risk of translation or rotation once deployed. In one embodiment, the implant 212 with the distal anchor 202 may be placed in the inferior vena cava as illustrated by FIGS. 11A and 11B. As such, clinicians may be able to position the implant within the vena cava without having to rely on still imagery or recorded video via CT scans or quantitative angiography. Instead, in an embodiment, the clinician may reference the anatomical features within the VC such as the intersection or junction between the renal vessels 220 that extend from the main trunk IVC 222 as an anatomical marker to identify where to position the implant 212. Here, the elongated distal anchor 202 may be positioned. FIGS. 11A and 11B provide illustration of the IVC as it extends below the heart of a patient along with the kidneys and associated renal vessels 220 that branch outwardly from the IVC 222. In one embodiment, the distal anchor 202 has an elongated and angled distal anchoring structure that may allow the implant 212 to self-correct its position within the vessel. The distal anchor 202 may have a generally J-hook shape and be positioned to extend deep into the right or left renal branch vessel of the IVC while the proximal anchor 204 may be positioned downstream or upstream from the branch 220 within the IVC 222. Here, the implant body may be positioned against an opposite wall of the IVC vessel than the renal branch vessel that the distal anchor 202 has been positioned. This may help prevent the implant from translating and rotating and allow the implant to be permanently located therein.

In another embodiment, the distal anchor 202 may be positioned via a delivery catheter in which the catheter may have a moveable end portion that can be toggled between a straight end portion and hook shaped end portion (not shown). The steerable end may be attached to the distal anchor 202 for placement within the anatomical marker or branch junction as desired. This may assist a clinician to identify the anatomical market for implant placement.

In the embodiment illustrated by FIG. 11B, the distal anchor 202′ may include a biasing feature in which the distal anchor 202′ is shaped to include a detent protrusion 260. The detent protrusion 260 may be retained in a collapsed configuration during implant delivery and, during positioning, may be released into an expanded position once the implant has been located near the anatomical feature. The detent protrusion 260 may slide along the interior vessel wall (such as the interior vessel wall of the IVC, SVC, or PA) until it reaches the anatomical marker or branch junction. Once the implant has been located adjacent the anatomical marker, the detent protrusion 260 may bias into the anatomical feature and abut against opposing walls within the anatomical feature to secure the implant in the desired location within the vessel. In this instance, the detent protrusion 260 may have a generally D shaped wire configured to fit snugly within the anatomical feature. FIG. 11B illustrates the detent protrusion positioned within a renal branch vessel but this configuration may be utilized in any other anatomical marker within the cardiovascular anatomy. Once the clinician feels the detent protrusion 260 spring or bias within the renal vessel or other branch vessel/anatomical feature, or views it fluoroscopically, the clinician may be signaled that the implant 212 is in the desired position. In both embodiments of FIGS. 11A and 11B, the implant 212 may be located within the vessel and arranged as identified in FIG. 10 to establish wireless communication with a device located outside the body.

Additionally, any embodiment of the anchors described herein may be configured to include any combination of features. In an embodiment, at least one anchor may also include a barb or hook 254 positioned along the anchor and extending therefrom to allow the anchor to attach along an inner surface of the anatomy.

FIGS. 12A-12D illustrate another embodiment in which the implant 212 and associated distal anchor 202 and proximal anchor 204 may be configured to be placed within the VC region along an anatomical feature such as the iliac vessels. In this embodiment, the distal anchor 202 and proximal anchor 204 may have elongated configurations that may be implanted within the vessel in a retracted position attached to a delivery catheter 502. One such delivery catheter is disclosed by commonly owned application Ser. No. 14/428,551 that incorporates release wires and various lumens therein to assist with the implantation. Here, the distal and proximal anchors may be sequentially expanded to position the implant 212 in at the anatomical marker. FIG. 12A illustrates a schematic version of the delivery catheter and anchors 202, 204 in the retracted position wherein the distal anchor 202 is attached to a distal end of the implant 212 and is collapsed over the longitudinal length of the implant body along the delivery catheter. The proximal anchor 204 is in the retracted position and extended proximally from the proximal end of the implant body along the delivery catheter. The implant 212 attached to the delivery catheter in the retracted position may be inserted within the IVC from one of the iliac branch vessels. The implant 212 and distal anchor 202 may be inserted such that end portions of the anchors may extend passed an apex branch 500 or Y intersection between the IVC and iliac vessels.

FIG. 12B illustrates the delivery catheter 502 and implant 212 within the IVC wherein the distal anchor 202 is released from the retracted position and the proximal anchor 204 remains in the retracted position. The distal anchor 202 may extend away from the delivery catheter 502 and abut against a vessel wall opposite from the iliac vessel in which the delivery catheter is located. Here, the distal anchor 202 remains partially extended over the longitudinal length of the implant body such that the end of the distal anchor 202 is angled towards the opposing vessel along an opposite side of the apex branch 500.

FIG. 12C illustrates the delivery catheter 502 and the implant within the IVC wherein the delivery catheter 502 is translated upstream within the IVC relative to the position in FIGS. 12A and 12B. This position causes the distal anchor 202 to be positioned along the opposing iliac vessel from the proximal anchor 204. The proximal anchor 204 is now released from the retracted position and is in the expanded position to abut against a vessel wall along the opposite iliac vessel from the distal anchor 202. The opposing locations of the anchors 202, 204 along the implant 212 are positioned near the apex 500 or Y intersection of the iliac vessels and IVC. FIG. 12D illustrates the implant 212 positioned in place with the delivery catheter 502 removed.

In the embodiments, both anchors 202/204 can be collapsed down and attached to a delivery catheter 502 via “release wires” or other mechanism. The implant 212 and anchors 202/204 can be introduced into the human vasculature in the collapsed position wherein removal of the release wires may allow the distal anchor to expand while the proximal anchor remains retracted such that the implant may be translated or rotated to configure to correct location of the implant and anchors within the anatomical features of the vessels. Further, removal of the release wires may allow subsequently allow the proximal anchor to be released from the retracted position once the desired location of the implant and anchors has been established.

In a further embodiment, the implant in FIGS. 12A-12D may feature a third anchor 280 (FIG. 12D) that extends distally from the housing of the biomedical sensor 212. The addition of a third anchor is optional but may assist to place the housing at a direction more proximally on the catheter than depicted in FIG. 12A, and the third anchor 280 would be attached or tied down to the catheter distally related to the housing. It would deploy when the implant is in the position shown in FIG. 12A, and would deploy before the other two anchors are deployed and prior to deployment of anchor 202 in FIG. 12B. On deployment, the third anchor would extend upward into the IVC, so that each of the common iliac veins and the IVC has a long anchor extending a sufficient distance to prevent the implant from turning the Y-junction corner sufficiently to enter the IVC.

FIG. 13 illustrates a similar concept as disclosed by FIGS. 12A-12D but the implant 212 and anchors 202, 204 are positioned within the SVC. In this example embodiment, the anatomical feature that the separately releasing anchors may span is between the brachiocephalic vessel and the internal jugular vessel. However, the delivery method described may allow the position and implantation of implants at various anatomical features or branch joints within the cardiovascular system of a mammal. Notably, in each embodiment, a contrast agent may be introduced to the blood flow to allow a clinician to view the location of the implant with imaging equipment. Due to the nature of blood flow within the CVS the fluoroscopic contrast agent may not provide sufficient implant and anchor identification within smaller branch vessels as the blood flow is directed towards the heart and not towards smaller branch vessels. The clinician may introduce such contrast agent within the vessel in which the distal anchor 202 may be placed, upstream of the target placement location, for example from a Peripherally Inserted Central Catheter (PICC). FIG. 13 illustrates that contract may be also introduced into the internal jugular vessel or the subclavian vessel to allow for implantation in the SVC, either from a lumen in the delivery system (as labeled in the Figure) or via another PICC. However, various vessels may be utilized to introduce contrast agents from an upstream location to assist a clinician with visualization.

The assembly, method and system disclosed allow for ease of implant placement as the distal anchor 202 may configured so that when the catheter is removed, there is very little chance of migration into an alternate vessel or alternate branches of the IVC or SVC. The embodiments may also provide an anatomical landmark facilitating location of the target implant site that may be easily identified by basic angiography and may allow a clinician to align the implant such that the distal anchor and proximal anchor span connecting branch vessels to position the implant in the CVS. The disclosure may further prevent unwanted rotation due to: the spring force nature of the anchor, delivery system rubbing against the implant during removal, and patient coughing or other patient movement. The angle that the target veins make with respect to the chest skin surface may ensure that the implant assumes an angle towards the chest that is optimal for RF or other wireless communication. The angle may ensure that the implant faces the chest surface when the distal anchor 202 is placed into the opposing branch of the respective vessel within the VC.

Further, if there is an unintentional deployment that is too distally positioned in the IVC or SVC, the distal anchor 202 may still fit within the opposing branch segment. If there is an unintentional deployment too proximally positioned relative to the heart, the distal anchor 202 may act to “pull” the implant 212 in the distal direction. In the event that the proximal anchor 204 may migrate due to the spring force action, the turn of the distal anchor 202 in the branch segment may prevent it from translating as the elongated distal anchor will be generally prevented from “turning the corner” as the device moves proximally. Further, if there is migration of the implant 212 distally, the housing and proximal anchor 202 may form an angle that prohibits them from making the turn. As such, the implant 212 includes self-adjusting properties in this anatomical location within the VC.

The two anchors may act to hold the bottom surface 62 of the implant 212 against the vessel wall with the sensor 40 and top surface 60 away from the vessel wall. The distal anchor may utilize its length relative to the implant to prevent rotation, by staying in the downturn or branch vessel. Additionally, it may prevent unintended interactions with other branches of the CVS. The distal anchor 202 may not include loops and may be too straight and long to migrate into side branches easily.

In one embodiment, the implant 212′ of FIG. 13 may be considered a second biomedical sensor 212′ as described herein and which may be configured to wirelessly communicate with either an external device or with another of the biomedical sensors or implants 212 as described herein. For example, the second biomedical sensor 212′ may be positioned along a downstream or outlet side of a heart of the patient such as in the pulmonary artery. The first or other biomedical sensor 212 may be positioned along an input side such as within the central venous system of the cardio vasculature and as illustrated by FIGS. 11A-12D. In such a configuration, the first and second biomedical sensors 212, 212′ may communicate various pressures at both an inlet area (upstream) and an outlet area (downstream) of the right heart of a patient. In a similar embodiment, a sensor in the pulmonary vein and another in the arterial system may provide input and output pressures of the left heart. Other combinations of sensor locations may be envisioned by those skilled in the art to provide simultaneous readings that provide clinically valuable insight into the status of portions of the cardiovascular system. The readings or measurements taken by the first and second biomedical sensors may be compared either by the use of a reader system or manually by a clinician to determine if different pressure readings by the first and second sensors would provide signs of heart conditions and whether there is a need to apply therapy to the patient to correct the condition. In the case of sensors that transduce a parameter into a resonance frequency, each sensor could be tuned to a different frequency range to prevent crosstalk and identify sensor location.

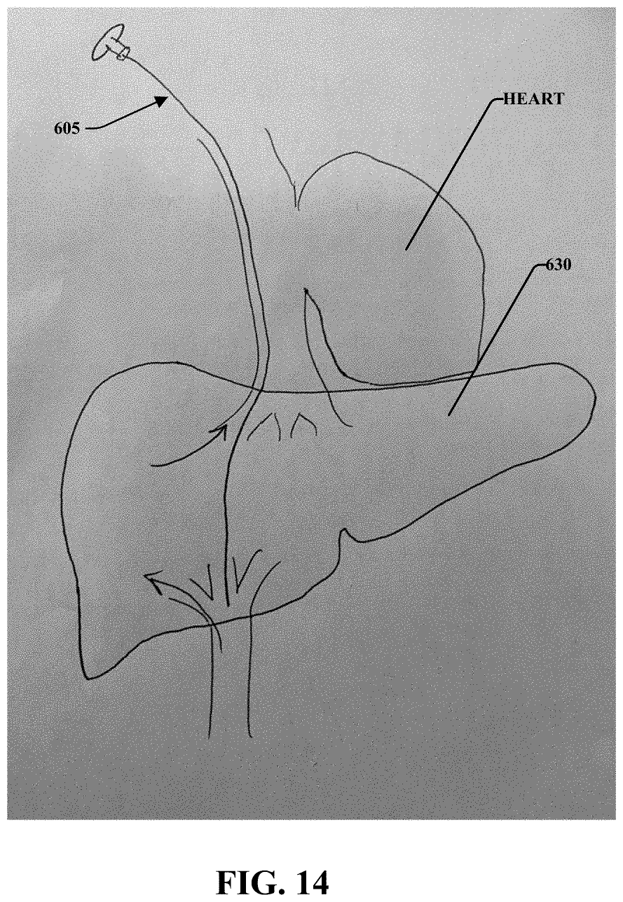

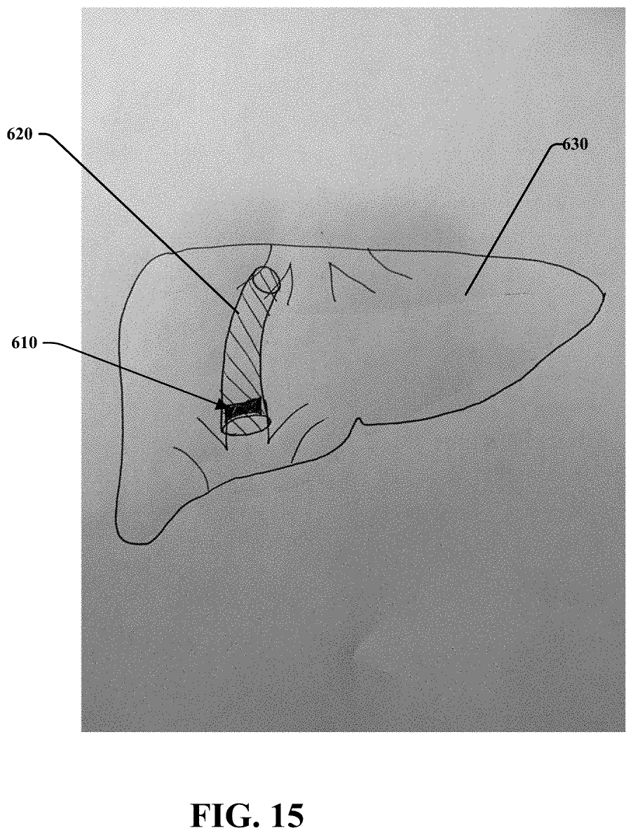

FIGS. 14 and 15 illustrate use of a biomedical sensor and anchoring system as described herein along with a stent or shunt in an organ. Notably, a transjugular intrahepatic portosystemic shunt (TIPS) is a device placed in the liver or other organ. The shunt is a generally hollow tube or stent that is placed within a vessel or other anatomy to bypass the liver or other organ. The tube is configured to provide fluid communication or otherwise connect an inlet vein, such as the portal vein, directly to an outlet vein, such as the hepatic vein. This device is primarily used for organs that are unable to properly function by receiving blood flow from an inlet vein and properly communicating it to its outlet vein.

Use of TIPS has been known to assist with cirrhosis of the liver and other liver issues. Thus it is also desirable to place an implant such as a biomedical sensor 610 within the shunt 620 along a position that is adjacent to an inlet vein such as the portal vein of a patient when the TIPS system has been implanted. Here, the anchoring system may include a biomedical sensor 610 and an anchoring system comprising an elongated hollow tube 620 having a distal end and a proximal end, where the biomedical sensor 610 is configured to be attached within the elongated hollow tube, and the tube serves as the shunt.

The proximal end of the elongated hollow tube 620 may be configured to be in communication with a perimeter of an organ 630 and the distal end of the elongated hollow tube is configured to be in communication with an opposite perimeter of said organ. The elongated hollow tube 620 is configured to allow fluid communication between the perimeter and the opposite perimeter. In one embodiment, the organ 630 is a liver and the elongated hollow tube 620 is a stent that is configured to establish fluid communication between a portal vein and a hepatic vein. The biomedical sensor 610 may be configured to measure and wirelessly communicate at least one of the following data points to an external reader device: pressure, temperature, GPS location, time, elevation, PH level, flow rate, and signal strength.

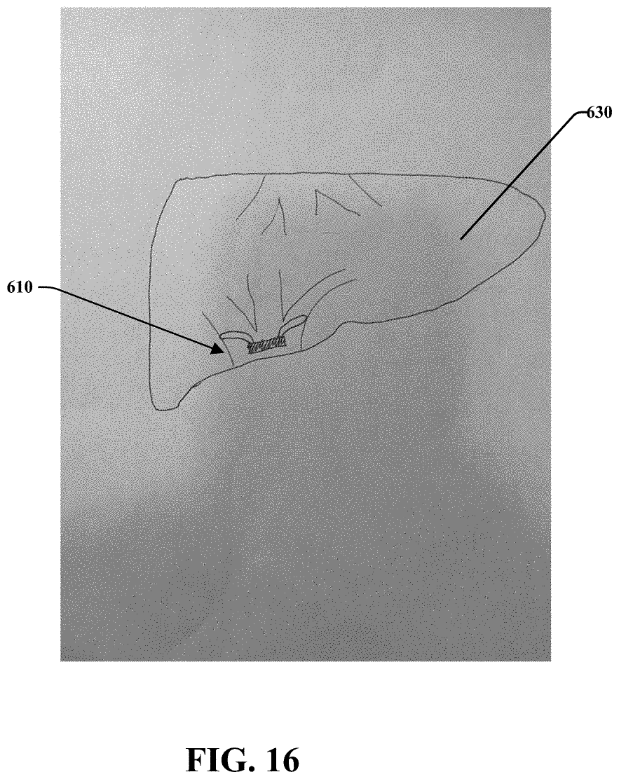

In a further embodiment as illustrated by FIG. 16, the biomedical sensor 610 may be implanted directly into a transplanted organ after it has been explanted from the donor and before it is implanted into a receiving patient. In the case where the organ is a liver, the sensor may be pre-implanted into the new liver's portal vein or into the vestigial portal vein of the receiving patient. In the case where the organ is a lung, the sensor may be pre-implanted in the new lung's pulmonary artery or the vestigial pulmonary artery of the patient. Other examples of pre-implanted transplanted organs with sensors will be obvious to those skilled in the art.

The TIPS application in addition to placing a sensor or other implant 610 on the shunt 620 itself or within the portal vein thereon may be applicable to a variety of applications. This may include but not be limited to organ transplants or organ related surgeries related to the heart, lungs, kidneys, spleen, stomach, pancreas, and joints such as hips and knees, as well as the liver. In one such usage, the biomedical sensor 610 as illustrated by FIG. 15 may be fastened to the stent 620 and placed within a subject organ (via guidewire 605 as illustrated by FIG. 14 and within an organ as illustrated by FIG. 15). The biomedical sensor may also be directly placed within or on a replacement organ (FIG. 16) prior to its transplant or other corrective surgical procedure to allow a medical practitioner to take remote wireless readings at a desired location within the subject organ before, during and after the transplant or surgical procedure.

In this way a physician or medical practitioner may check the functioning of the TIPS or shunt and receive an early warning when the inlet vein pressure such as the portal vein pressures are in danger of rising to unsafe levels. They could repair or replace the TIPS, or treat the patient with drugs or other therapy, when the first portal vein pressure increases occurred, indicating possible shunt narrowing. They could do this before hematemesis or other severe symptoms occur. Thus it is contemplated that the junction of the portal vein and hepatic vein may be an anatomical marker and desired location for implanting such implants and anchoring structures disclosed herein.

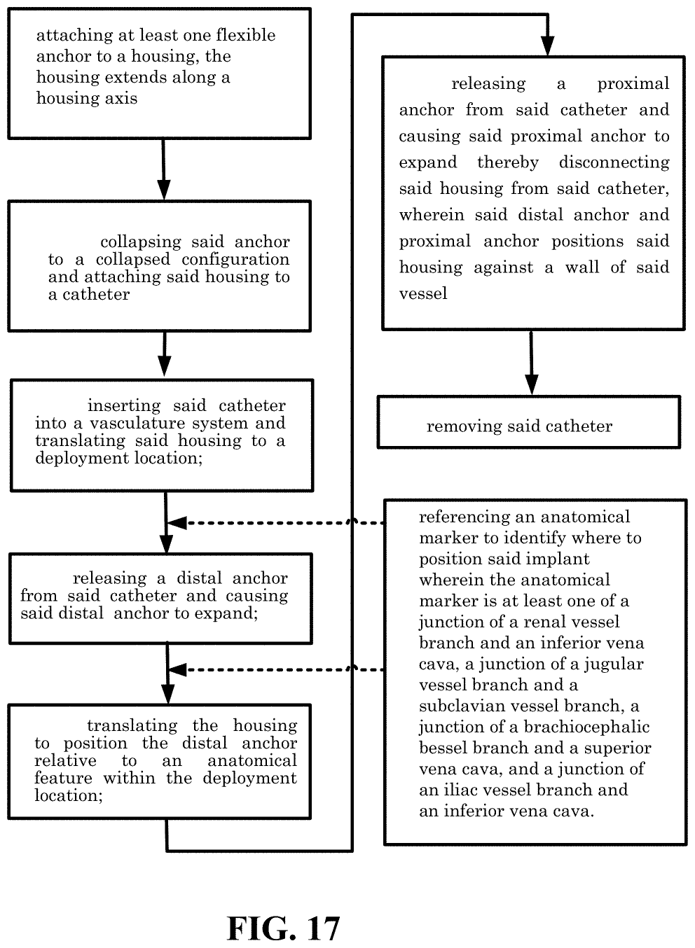

FIG. 17 illustrates a flow chart that describes the steps for a method for anchoring an implant inside a blood vessel, comprising the steps of: attaching at least one flexible anchor to a housing, the housing extends along a housing axis; collapsing said anchor to a collapsed configuration and attaching said anchors to a catheter; inserting said catheter into a vasculature system and translating said housing to a deployment location; releasing a distal anchor from said catheter and causing said distal anchor to expand; translating the housing to position the distal anchor relative to an anatomical feature within the deployment location; releasing a proximal anchor from said catheter and causing said proximal anchor to expand thereby disconnecting said housing from said catheter, wherein said distal anchor and proximal anchor positions said housing against a wall of said vessel; and removing said catheter.

This method may also include the step of referencing an anatomical marker to identify where to position said implant. The anatomical marker is a junction of a renal vein and an inferior vena cava, a junction of a jugular vein branch and a subclavian vein branch, a junction of a brachiocephalic vein branch and a superior vena cava, or a junction of an iliac vein branch and an inferior vena cava.

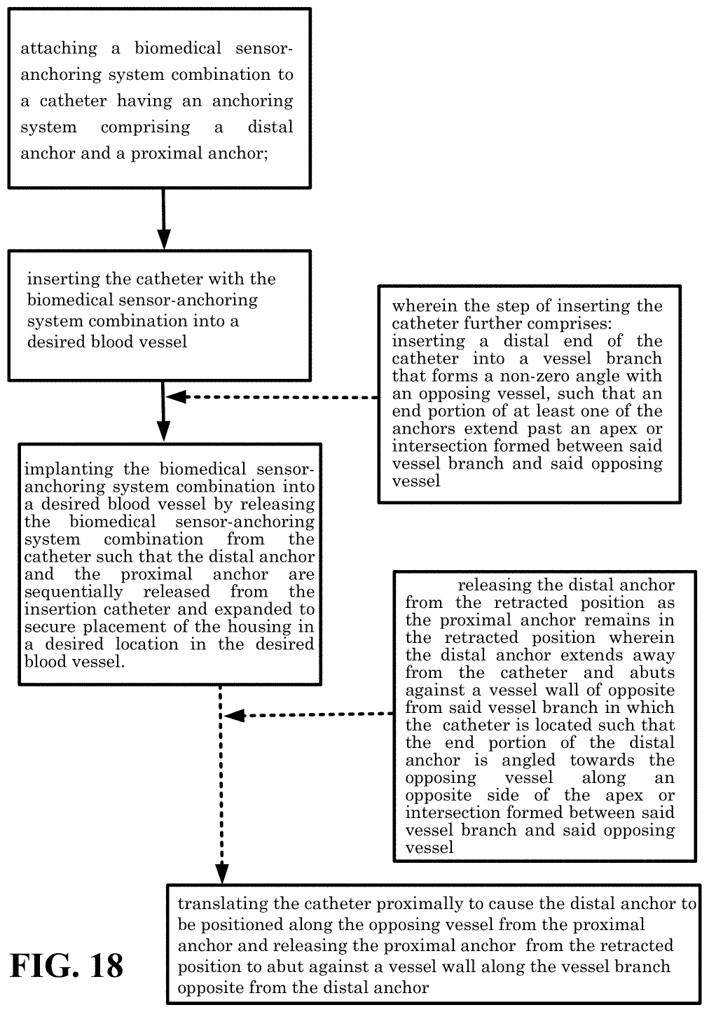

FIG. 18 illustrates a flow chart that describes the steps for a method for inserting a biomedical sensor and anchoring system for securing same, the method comprising the steps of: attaching a biomedical sensor-anchoring system combination to a catheter where the biomedical sensor-anchoring system combination comprises: a biomedical sensor having a housing with a distal end and a proximal end; and an anchoring system comprising a distal anchor and a proximal anchor, where the distal anchor is attached to the distal end of the housing and the proximal anchor is attached to the proximal end of housing. At least one of the distal anchor or the proximal anchor has formed therein an elongated wire structure placed in a retracted position against the catheter so as to accomplish secure placement of the biomedical sensor upon implantation thereof; inserting the catheter with the biomedical sensor-anchoring system combination into a desired blood vessel; and releasing the biomedical sensor-anchoring system combination from the catheter such that the distal anchor and the proximal anchor are sequentially released from the insertion catheter and expanded to secure placement of the housing in a desired location in the desired blood vessel.

In this embodiment, the distal anchor may be collapsed over a longitudinal length of the biomedical sensor along the catheter and the proximal anchor is in the retracted position and extended proximally from the proximal end of the biomedical sensor. Also, the step of inserting the catheter further comprises: inserting a distal end of the catheter into a vessel branch that forms a non-zero angle with an opposing vessel, such that an end portion of at least one of the anchors extend past an apex or intersection formed between said vessel branch and said opposing vessel. Non-zero angle can be any angle between about 0 degree to about 180 degrees (more particularly between 1 degree and 179 degrees) wherein there exists a bend at an apex at the intersection between the vessel branch and the opposing vessel.

The distal anchor may be released from the retracted position as the proximal anchor remains in the retracted position wherein the distal anchor extends away from the catheter and abut against a vessel wall that is opposite from said vessel branch in which the catheter is located such that the end portion of the distal anchor is angled towards the opposing vessel along an opposite side of the apex or intersection formed between said vessel branch and said opposing vessel. The catheter may then be translated proximally (towards its origin) to cause the distal anchor to be positioned along the opposing vessel from the proximal anchor and releasing the proximal anchor from the retracted position to abut against a vessel wall along the vessel branch opposite from the distal anchor.

A third anchor may be positioned distally to the distal anchor and the proximal anchor when positioned in the retracted position along the catheter, said third anchor is configured to be released before the distal anchor and the proximal anchor and is configured to extend lengthwise into the inferior vena cava vessel wherein the biomedical sensor-anchoring system combination is configured to prevent migration of the biomedical sensor by inhibiting its ability to migrate within said vessel branch that forms a non-zero angle with the opposing vessel and the inferior vena cava vessel.

In another embodiment, a second catheter with a second biomedical sensor-anchoring system may be placed into a desired blood vessel wherein the biomedical sensor and second biomedical sensor are configured to wirelessly communicate with a device outside a chest of a patient.

While in accordance with the patent statutes the best mode and certain embodiments of the disclosure have been set forth, the scope of the disclosure is not limited thereto, but rather by the scope of the attached. As such, other variants within the spirit and scope of this disclosure are possible and will present themselves to those skilled in the art.

What is claimed is:

1. An anchoring system for a biomedical sensor comprising:
a biomedical sensor having a housing with a distal end and a proximal end; and
an anchoring system comprising a distal anchor and a proximal anchor, where the distal anchor is attached to the distal end of the biomedical sensor and the proximal anchor is attached to the proximal end of the biomedical sensor,
wherein at least one of the distal anchor or the proximal anchor is formed with an elongated flexible structure so as to accomplish secure placement of the biomedical sensor upon implantation thereof by a catheter device;
wherein said at least one distal and proximal anchors is configured to be placed into a retracted position for catheter delivery, and placed in an expanded position for placement within a vessel;
wherein said at least one anchor is configured to position said housing against a vessel wall, and;
wherein said at least one anchor is configured to adapt to at least one anatomical feature of a vessel to prevent movement of said housing.

2. The anchoring system of claim 1, wherein the biomedical sensor is configured to be implanted in a central venous vessel and the biomedical sensor is designed to be read from the chest of a patient in which the sensor is implanted.

3. The anchoring system of claim 1 wherein said at least one anchor is a wire.

4. The anchoring system of claim 3 wherein said wire is formed as a generally elongated loop shaped to conform to an inner surface of a vessel.

5. The anchoring system of claim 1 wherein said at least one anatomical feature is a first vessel segment oriented at an angle with respect to an adjoining second vessel segment and the housing includes a sensor surface configured to be positioned along an axis that is positioned towards a patients chest and configured to communicate with a wireless sensor reader device.

6. The anchoring system of claim 5 wherein said first vessel segment is a renal vein and said second vessel segment is the inferior vena cava.

7. The anchoring system of claim 5 wherein said housing is configured to be located in said first vessel segment, and said at least one anchor is configured to extend into said second vessel segment a distance sufficient to prevent translational or rotational movement of said housing in at least one direction by impeding movement of the housing about said angle formed by said vessel segments.

8. The anchoring system of claim 7 further comprising a second biomedical sensor configured to be located in a third vessel segment.

9. The anchoring system of claim 8 wherein said biomedical sensor and said second biomedical sensor are configured to communicate wirelessly with each other or with a wireless sensor reader device positioned outside said vessel containing said biomedical sensor and said second biomedical sensor.

10. The anchoring system of claim 1 wherein said at least one anatomical feature is an intersection of the iliac vessels and the inferior vena cava vessel.

11. A method for anchoring an implant inside a blood vessel, comprising the steps of:
attaching at least one flexible anchor to a housing, the housing extends along a housing axis;
collapsing said anchor to a collapsed configuration and attaching said housing to a catheter;
inserting said catheter into a vasculature system and translating said housing to a deployment location;
releasing a distal anchor from said catheter and causing said distal anchor to expand;
translating the housing to position the distal anchor relative to an anatomical feature within the deployment location;
releasing a proximal anchor from said catheter and causing said proximal anchor to expand thereby disconnecting said housing from said catheter, wherein said distal anchor and proximal anchor positions said housing against a wall of said vessel; and
removing said catheter.

12. The method of claim 11 further comprising the step of referencing an anatomical marker to identify where to position said implant.

13. The method of claim 12, wherein said anatomical marker is a junction of a renal vein and an inferior vena cava.

14. The method of claim 12, wherein said anatomical marker is a junction of a jugular vein branch and a subclavian vein branch.

15. The method of claim 12, wherein said anatomical marker is a junction of a brachiocephalic vein branch and a superior vena cava.

16. The method of claim 12, wherein said anatomical marker is a junction of an iliac vein branch and an inferior vena cava.

17. A method for inserting a biomedical sensor and anchoring system for securing same, the method comprising the steps of:
(i) attaching a biomedical sensor-anchoring system combination to a catheter where the biomedical sensor-anchoring system combination comprises:
a biomedical sensor having a housing with a distal end and a proximal end; and
an anchoring system comprising a distal anchor and a proximal anchor, where the distal anchor is attached to the distal end of the housing and the proximal anchor is attached to the proximal end of housing,
wherein at least one of the distal anchor or the proximal anchor has formed therein an elongated wire structure placed in a retracted position against the catheter so as to accomplish secure placement of the biomedical sensor upon implantation thereof;
(ii) inserting the catheter with the biomedical sensor-anchoring system combination into a desired blood vessel; and
(iii) implanting the biomedical sensor-anchoring system combination into a desired blood vessel by releasing the biomedical sensor-anchoring system combination from the catheter such that the distal anchor and the proximal anchor are sequentially released from the insertion catheter and expanded to secure placement of the housing in a desired location in the desired blood vessel.

18. The method of claim 17 wherein the distal anchor is collapsed over a longitudinal length of the biomedical sensor along the catheter and the proximal anchor is in the retracted position and extended proximally from the proximal end of the biomedical sensor, wherein the step of inserting the catheter further comprises:
inserting a distal end of the catheter into a vessel branch that forms a non-zero angle with an opposing vessel, such that an end portion of at least one of the anchors extend past an apex or intersection formed between said vessel branch and said opposing vessel.

19. The method of claim 18, wherein the step of implanting the biomedical sensor further comprises releasing the distal anchor from the retracted position as the proximal anchor remains in the retracted position wherein the distal anchor extends away from the catheter and abuts against a vessel wall of opposite from said vessel branch in which the catheter is located such that the end portion of the distal anchor is angled towards the opposing vessel along an opposite side of the apex or intersection formed between said vessel branch and said opposing vessel.

20. The method of claim 19, wherein the step of implanting the biomedical sensor further comprises translating the catheter proximally to cause the distal anchor to be positioned along the opposing vessel from the proximal anchor and releasing the proximal anchor from the retracted position to abut against a vessel wall along the vessel branch opposite from the distal anchor.

21. The method of claim 20, further comprising a third anchor positioned distally to the distal anchor and the proximal anchor when positioned in the retracted position along the catheter, said third anchor is configured to be released before the distal anchor and the proximal anchor and is configured to extend lengthwise into an inferior vena cava vessel wherein the biomedical sensor-anchoring system combination is configured to prevent migration of the biomedical sensor by inhibiting its ability to migrate within said vessel branch that forms a non-zero angle with the opposing vessel and the inferior vena cava vessel.

22. The method of claim 17, further comprising inserting a second catheter with a second biomedical sensor-anchoring system into a desired blood vessel.

23. The method of claim 22, wherein the biomedical sensor and second biomedical sensor are configured to wirelessly communicate with a device outside a chest of a patient.

24. An anchoring system for a biomedical sensor comprising:
a biomedical sensor; and
an anchoring system comprising an elongated hollow tube having a distal end and a proximal end, where the biomedical sensor is configured to be attached within the elongated hollow tube,
wherein the biomedical sensor is positioned adjacent to at least one of the distal end or the proximal end of the elongated hollow tube; and
wherein the elongated hollow tube is configured to be inserted into an organ of a patient.

25. The anchoring system of claim 24, wherein the proximal end of the elongated hollow tube is configured to be in communication with a fluid inlet of an organ and the distal end of the elongated hollow tube is configured to be in communication with a fluid outlet of said organ; wherein the elongated hollow tube allows for fluid communication between the inlet and the outlet to allow for fluid bypass of the organ.

26. The anchoring system of claim 24, wherein the organ is a liver and the elongated hollow tube is a shunt between a portal vein and a hepatic vein.

27. The anchoring system of claim 24, wherein said biomedical sensor is configured to wirelessly communicate at least one of the following data points: pressure, temperature, GPS location, time, elevation, acidity, salinity, chemical composition, flow rate, and signal strength.

28. A method for implanting a biomedical sensor into an organ to be transplanted into a patient, the method comprising the steps of:
(i) explanting the organ from a donor;
(ii) implanting the organ with a biomedical sensor, wherein said biomedical sensor is a wireless sensor configured to communicate wirelessly with a wireless reader device;
(iii) surgically implanting the organ with the biomedical sensor into a receiving patient.

29. The method of claim 28, wherein said biomedical sensor is implanted into a vasculature system of the organ using a catheter.

30. The method of claim 29, wherein said biomedical sensor is provided with at least two anchors configured to extend into a vessel branch of said vasculature system wherein said vessel branch that forms a non-zero angle with an opposing vessel, such that an end portion of at least one of the anchors extend past an apex or intersection formed between said vessel branch and said opposing vessel, said anchors being collapsible when tied to said catheter and releasable from said catheter.

31. The method of claim 28, wherein said biomedical sensor is implanted into said organ by direct suture or staple.

32. The method of claim 28, wherein said biomedical sensor is implanted into a vestigial vasculature of a receiving patient near the point where it will connect to said transplanted organ, prior to implantation of said transplanted organ.

33. The method of claim 28 wherein said organ is selected from the following:
heart, lung, kidney, spleen, stomach, pancreas, heart, skeletal joints, and liver.
